(19) United States
(12) Patent Application Publication
(10) Pub. No.: US 2023/0285491 A1
(43) Pub. Date: Sep. 14, 2023

(54) COMPOSITION FOR PREVENTION OF DUMPING SYNDROME IN A POST-BARIATRIC SURGERY SETTING

(71) Applicant: METAGENICS, INC., Aliso Viejo, CA (US)

(72) Inventors: An-Katrien VYNCKIER, Oostende (BE); Lise VAN WYNGENE, Oostende (BE); Mieke VAN DEN DRIESSCHE, Oostende (BE)

(21) Appl. No.: 18/016,692

(22) PCT Filed: Jul. 23, 2021
(86) PCT No.: PCT/US2021/042861

§ 371 Date: Jan. 18, 2023

Related U.S. Application Data
(60) Provisional application No. 63/055,607, filed on Jul. 23, 2020.

(51) Int. Cl. A61K 36/605; A61K 47/36; A61P 1/08; A61K 9/00

(52) U.S. Cl. CPC A61K 36/605; A61K 9/0056; A61K 47/36; A61P 1/08

(57) ABSTRACT

This disclosure provides a composition for the improved method of treating, preventing and controlling dumping syndrome (“DS”), whether early dumping or late dumping, commonly encountered in surgery of the GI tract, especially in bariatric surgery, such as in Roux-en-Y gastric bypass and vertical sleeve gastrectomy. In general, the composition is in the form of an oral composition for the treatment of dumping syndrome. The oral composition comprises: a) an alpha-glucosidase inhibiting component comprising an extract of Morus alba; and b) a glucagon-like peptide (GLP-1) inhibiting component comprising a dipeptidyl peptidase-4 (DPP-4) enzyme or fragment thereof.

### CROSS-REFERENCE TO RELATED APPLICATIONS

This application claims priority to and all advantages of U.S. Provisional Pat. Application No. 63/055,607 filed on 23 Jul. 2021, the content of which is incorporated herein by reference.

### FIELD OF THE INVENTION

This invention relates to treatments for dumping syndrome, and to a composition for use in that treatment.

### BACKGROUND OF THE INVENTION

Dumping syndrome (“DS”) is a frequent complication of esophageal, gastric or bariatric surgery. DS occurs in up to 40% of patients after Roux-en-Y gastric bypass surgery or sleeve gastrectomy. Symptoms of DS may occur shortly after surgery and can persist for years if left untreated. For example, one study suggested that DS is present in 19% of patients 2.5 years after Roux-en-Y gastric bypass surgery. Another study found symptoms suggestive of DS in 33% of patients one year after sleeve gastrectomy surgery. Rapid gastric emptying, with the delivery to the small intestine of a significant proportion of solid food as large particles that are difficult to digest, is a key event in the pathogenesis of this syndrome. Rapid gastric emptying causes a shift of fluid to the intestinal lumen, which results in cardiovascular symptoms, release of several gastrointestinal and pancreatic hormones, and late postprandial hypoglycemia.

Early dumping symptoms comprise both gastrointestinal and vasomotor symptoms. Symptoms happen between 30 and 60 minutes after meals as a result of rapid instillation of meals into the small bowel and decreased blood volume due to intraluminal fluid sequestration. Most symptoms are abdominal (e.g., bloating and pain) and systemic (e.g., palpitations, fatigue, tachycardia, lightheadedness, and syncope).

Late dumping symptoms are the result of reactive hypoglycemia. The most commonly observed symptoms and signs are due to neuroglycopenia and hyperadrenergic state (e.g., decreased consciousness, shakiness, and difficulty concentrating). It is important to note that often patients may present both early and late DS at the same meal. DS reduces the quality of life as symptoms are usually severe and can limit sports capacity and everyday activities.

Conventional treatment of dumping occurs in stages. Since either early or late dumping may occur in a patient population, each symptom tends to be treated individually. The net result is a “stepwise” treatment of the malady. The first step in conventionally treating DS is the introduction of dietary measures, such as small and frequent meals and inclusion of fiber and protein in every meal. In this intervention, regardless of whether there is early dumping or late dumping, the patient is assured of some benefit from each small meal. The use of guar gum and pectin, for example, can slow gastric emptying.

In some cases, acarbose, a glucosidase inhibitor that slows carbohydrate digestion, can be added to these measures for patients with hypoglycemia. The rationale of protection of late dumping by acarbose is based on the fact that this drug delays glucose absorption.

Somatostatin analogs are thought to be the most effective medical therapy for DS. Somatostatin analogs are typically used on patients who have failed to respond to other therapies and whose symptoms are markedly expressed. A slow-release preparation is the treatment of choice, because of the preparation’s ease of administration and superior effect on quality of life.

In patients with treatment-refractory DS, surgical reintervention or continuous enteral feeding can be considered. However, the outcomes of such approaches are variable.

Recently, a novel diagnostic and therapeutic algorithm was proposed based on the international Delphi consensus process. In this algorithm, a systematic guideline is described in order to provide rapid DS diagnosis and start-up possible treatment options (see, e.g. FIG. 1). In short, in patients with a history of gastric surgery, the presence of symptoms suggestive of DS should raise clinical suspicion. Other reasons for these symptoms should be excluded by standard diagnostic evaluation and a modified glucose tolerance test is preferred to confirm the diagnosis of DS. Dietary measures should be undertaken as a preferential initial approach. Next, in patients that do not respond to diet modifications, the use of acarbose is recommended. Alternatively, patients that do not respond to the above interventions can be treated with somatostatin analogues. Lastly, other experimental approaches such as surgical re-intervention and pancreatic resection have to be carefully considered.

Glucagon-like peptide (GLP)-1 is a peptide hormone that is produced by the epithelial endocrine L-cells of the intestine in response to unabsorbed nutrients. Its main purpose is to act as an incretin hormone to stimulate the secretion of insulin by the beta cells of the pancreas in response to meal intake. Strong increases in levels of circulating GLP-1 have been observed in bariatric patients after meal ingestion. In fact, it is believed that the type of bariatric surgery influences the magnitude of the GLP-1 increase as absolute concentrations of the hormone were shown to be nearly 10 times higher after Roux-en-Y gastric bypass and vertical sleeve gastrectomy, compared to patients with a gastric banding. This phenomenon can be explained by the fact that in these groups of patients gastric emptying is accelerated, resulting in increased quantities of undigested nutrients in the intestine and exaggerated GLP-1 and insulin secretion.

REDUCOSE® and similar compounds lower postprandial blood glucose and postprandial blood insulin following a carbohydrate challenge. REDUCOSE® lowers the postprandial blood glucose levels by up to 42% (p<0.001). REDUCOSE® is also known to lower the postprandial insulin response by a corresponding amount (-41%, p<0.001). No adverse effects are known and there are no known differences between REDUCOSE® and placebo in incidence or severity of GI side-effects.

Hypoglycemia, the main symptom of the late DS, is driven by a rapid GLP-1 release and hyperinsulinemic response. Targeting the rapid GLP-1 secretion is therefore a promising strategy to alleviate hypoglycemia and late DS in bariatric patients.

It is believed that the inhibition of GLP-1 signaling via administration of the GLP-1 receptor antagonist Exendin-(9-39) corrected postprandial hypoglycemic and hyperinsulinemic events in gastric bypass patients. This inhibition effect was also observed where GLP-1 receptor antagonism prevented hypoglycemia in all patients and reversed neuroglycopenic symptoms. Thus, it is believed that targeting of the GLP-1 signaling pathway successfully alleviates postprandial hypoglycemia.

Efficacy of agents that increase meal viscosity, such as guar gum, pectin and glucomannan, are generally understood to mitigate DS in patients with DS. The rationale for their use is that increasing the consistency of the meal slows the rate of gastric emptying and therefore also the release of nutrients into the intestine. Consequently, thickening agents (or viscosity enhancing components) should reduce the shift of fluid from the intravascular component to the intestinal lumen due to hyperosmolarity, and reduce the release of intestinal incretin hormones, thereby also improving postprandial hypoglycemia.

Several studies administering pectin, guar gum or glucomannan in dosages ranging from 1.3 g to 15 g together with each meal have shown that these thickening agents slow gastric emptying, reduce the release of gastrointestinal hormones (insulin and gastric inhibitory polypeptide), and control symptoms of DS. Additionally, the delayed exposure of the intestine to unabsorbed nutrients might reduce the release of GLP-1 by the secretory L-cells and contribute to the improvement of hypoglycemia and dumping symptoms.

### Problem to Be Solved

There is currently a lack of a treatment that reduces or eliminates dumping syndromes, whether late dumping or early dumping, without a “stagewise” method of treatment. This modulating of gastromotility would improve the quality of life and benefit patients suffering therefrom.

### SUMMARY OF THE INVENTION

This disclosure provides a novel medical food or supplement composition that treats dumping syndrome. This disclosure also provides a composition offering a multi-faceted approach to target the main causes of dumping in post-surgery bariatric patients by targeting the hypoglycemia and the hyperosmolarity in the jejunum as well as the act on the release of the incretin GLP-1. In this way, the composition provided treats both late dumping and early dumping symptoms without requiring a “stagewise” method of treatment. The composition typically comprises DPP-4, a glucosidase inhibiting portion, and additional other components as desired. The use of the composition as defined below treats dumping syndrome.

In various embodiments, an oral composition for the treatment of dumping syndrome is provided. The oral composition comprises: a) an alpha-glucosidase inhibiting component comprising an extract of Morus alba; and b) a glucagon-like peptide (GLP-1) inhibiting component comprising a dipeptidyl peptidase-4 (DPP-4) enzyme or fragment thereof.

### BRIEF DESCRIPTION OF THE DRAWINGS

FIG. 1 is an example of a diagnostic and treatment algorithm for dumping syndrome.

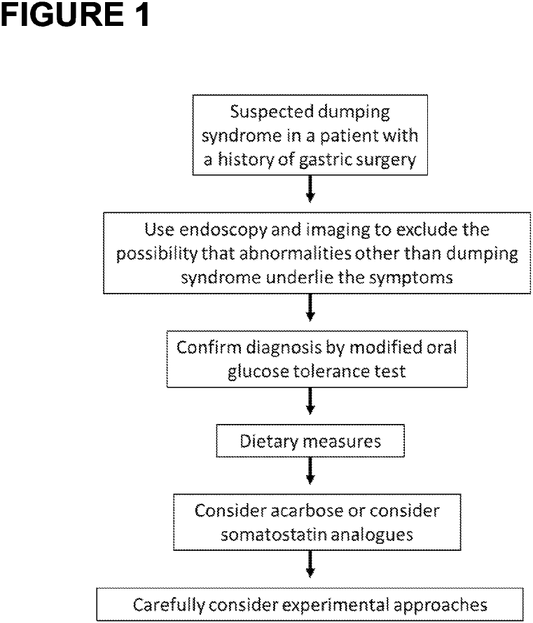

### DETAILED DESCRIPTION OF THE INVENTION

This disclosure provides a composition offering a multi-faceted approach to target the main causes of dumping. In general, the composition is in the form of an oral composition.

The oral composition for the treatment of dumping syndrome (“DS”) disclosed herein comprises an alpha-glucosidase inhibiting component and a glucagon-like peptide (“GLP-1”) inhibiting component. Typically, the alpha-glucosidase component comprises, alternatively consists of, an extract of Morus alba. Typically, the GLP-1 inhibiting component comprises, alternatively consists of, a dipeptidyl peptidase-4 (“DPP-4” or “DPP-IV”) enzyme or fragment thereof. In various embodiments, the oral composition consists essentially of, or consists of, the alpha-glucosidase inhibiting component and the GLP-1 inhibiting component.

In some embodiments, the oral composition for the treatment of DS comprises a GLP-1 inhibiting component. Typically, the GLP-1 inhibiting component comprises a dipeptidyl peptidase-4 (DPP-4) enzyme, or an active fragment thereof. Without being bound to this theory, it is likely that the GLP-1 inhibiting component tackles the effect of released incretin, GLP-1, by causing a degradation and thus inactivation of the GLP-1.

Typically, the amount of the GLP-1 inhibiting component (dose range) is from about 300 to 60,000 DPP-4 units (DPP-4 activity/g), alternatively 500 to 50,000 DPP-4 units (DPP-4 activity/g), or alternatively 1,000 to 10,000 DPP-4 units (DPP-4 activity/g). One unit of DPP-4 is the amount of enzyme that will hydrolyze the DPP-4 substrate to yield 1.0 mmole of AMC per minute at 37° C.

The skilled artisan can assay this activity, whether of a whole enzyme or a fragment thereof, to determine the effective amount via a commercially available product, for example, a Sigma-Aldrich DPP4 Activity Assay Kit Catalog Number MAK088. One of skill in the art would be able to assay this activity by using commercially available products, such as Promega’s DPPIV-GLO Protease assay G8350/8350 (Promega, Madison WI). Without being bound by this theory, the rapid and effective degradation and inactivation of GLP-1 by DPP-4 addresses exaggerated postprandial GLP-1 levels seen in bariatric patients.

Use of DPP-4 in the oral composition of this disclosure, and use thereof closely before (e.g., 30-60 minutes before, or alternatively 60-120 minutes before) or during meal ingestion avoids the hyperinsulinemic and hypoglycemic events in bariatric patients. The incretin hormone GLP-1 is rapidly degraded by the DPP-4 enzyme, which is expressed in the enterocyte brush, but also in the endothelial cells lining the capillaries of the lamina propria. In addition, DPP-4 is found in circulation in a soluble form. Due to its rapid degradation by DPP-4, only small amounts of GLP-1 (i.e., 10-15%) reach circulation under physiological conditions. Importantly, endogenous DPP-4 activity was shown to be reduced by gastric bypass intervention in obese patients with type 2 diabetes.

In various embodiments, the oral composition for the treatment of DS comprises an alpha-glucosidase inhibiting component. Typically, the alpha-glucosidase inhibiting component comprises an extract of Mulberry (Morus alba). In certain embodiments, the extract of Morus alba is an aqueous extract of Morus alba leaf, alternatively Morus alba fruit, alternatively Morus alba stem, or alternatively Morus alba root.

In particular embodiments, the extract of Morus alba comprises an iminosugar in an amount of 2.5-15 mass%, alternatively 4-10 mass%, or alternatively 5-8 mass%. In specific embodiments, the extract of Morus alba is 5 mass% of the iminosugar 1-deoxynojirimycin (DNJ).

In certain embodiments, the alpha-glucosidase comprises, or alternatively consists of, CAS 94167-05-02. CAS 94167-05-02 has an activity standardized to one of the chemical components thereof. For example, REDUCOSE® is a proprietary extract of mulberry leaves that is standardized to contain 5% (m/m) 1-deoxynojirimycin (DNJ), a structural analog of D-glucose. DNJ is a reversible, competitive natural inhibitor of α-glucosidase enzymes, which is one of the main enzymes involved in breaking down carbohydrates in the gut to facilitate glucose absorption. In certain embodiments, the amount or dosage of the alpha-glucosidase in the oral composition of this disclosure is from about 125 mg to 500 mg, alternatively 200 mg to 300 mg, or alternatively 225 mg to 250 mg, per dose.

In various embodiments, the oral composition further comprises a viscosity enhancing component (or thickening agent). In particular embodiments, the viscosity enhancing component comprises a soluble dietary fiber. In certain embodiments, the viscosity enhancing component comprises, alternatively consists of, at least one component selected from the group consisting of gums (e.g. guar gum, carob seed gum, tic gum, etc.), fibers, pectins, polysaccharides (e.g. glucomannan, galactomannan, inulin and/or fructooligosaccharide, human milk oligosaccharide), starches (e.g. digestion resistant starches), and combinations thereof, to increase viscosity and moderate the early dumping symptoms. In certain embodiments, the oral composition comprises guar gum. In some embodiments, the amount or dosage of the viscosity enhancing component in the composition of this disclosure is from about 0.01 g to 20 g, alternatively 0.05 g to 15 g, or alternatively 0.05 g to 5 g, per dose.

In some embodiments, the oral composition further comprises a supplement component. Typically, the supplement component comprises compatible vitamins, minerals, and other supplements that may be added as desired. The skilled artisan will appreciate that bariatric patients, and those that cope with DS are generally predisposed to certain vitamin and mineral deficiencies. For example, deficiencies in calcium, phosphorus, iron, chrome, copper, iodine, manganese, magnesium, molybdenum, selenium, zinc, vitamin B12, vitamin C, vitamins B1, B2, B3, B5, B6, B9 and fat soluble vitamins (e.g., vitamins A, D, E, K) are common. In particular embodiments, the supplement component comprises, alternatively consists of, one of calcium, phosphorus, iron, chrome, copper, iodine, manganese, magnesium, molybdenum, selenium, zinc, vitamin B12, vitamin C, Vitamins B1, B2, B3, B5, B6, B9, fat soluble vitamins (e.g., vitamins A, D, E, K), or combinations thereof. To the extent these vitamins, minerals, and other supplements are combined with or utilized with the composition of this disclosure, the oral composition manages an increased number of symptoms and issues. Examples of other suitable supplemental components that may be used in or with the composition of this disclosure are described in WO2018017456A1 and WO2021127164A1, the disclosures of which are incorporated herein by reference.

### Dosage Forms of the Composition

The use of the oral composition for the manufacture of a medicament for treating or preventing DS is also disclosed. The oral composition may be provided in a capsule, a tablet, a powder or other forms understood in the art. Because of the low stomach volume of bariatric patients, it is typical that the dosage may be provided in small volume, typically in a capsule or pill form, if not comingled with food as via a powdered form.

In particular embodiments, the dosage is provided via an immediate release capsule. In certain embodiments, the oral composition is protected from gastric degradation. In specific embodiments, the oral composition is enterically coated. In other embodiments, the oral composition is free of an enteric coating. Examples of other suitable dosage forms for the composition of this disclosure are described in WO2018017456A1 and WO2021127164A1.

### Methods of Use

In some embodiments the components of the oral composition are provided prior to or immediately after a meal. Typically, the components of the oral composition are provided all together, either immediately prior to eating, or concurrent with the start of the eating. Such administration may occur by mixing the oral composition with a component of the meal, or by taking the dosage form at the start of the meal.

A method of treating or preventing DS is also disclosed. The method comprises providing the oral composition and orally administering the oral composition to a patient. In some embodiments, the oral composition is orally administered immediately before or with a meal. In specific embodiments, the oral composition is mixed with a component of a meal before being orally administered.

For example, if the oral composition is used in an immediate release capsule and taken at the time of a meal, preferably at the beginning of the meal, the oral composition would be active in the GI tract at the same time as the meal. Of course, the skilled artisan would be able to determine each patient’s optimal regimen. Examples of other suitable dosage/administrative regimes for the composition of this disclosure are described in WO2018017456A1 and WO2021127164A1.

The typical embodiment of the composition of this disclosure would provide the oral composition in an immediate release capsule taken immediately at the start of the meal. However, it should be appreciated that the composition is not limited to such a particular form.

### EXAMPLES

Without limiting the invention in any way, the following examples illustrate the invention’s uses, compositions, and methods, which together with the above description, illustrate the invention in a non-limiting fashion.

### Example 1: DPP-4 Dosage Determination

An observational study to determine a patient’s optimal DPP-4 dosage occurred is as follows: Patients ingest DPP-4 formulas with predetermined DPP-4 activity (ranging from 500 - 50,000 DPP-4 activity units), at the beginning of a standardized meal (typically a mixed-meal containing carbohydrates, protein, and fat (e.g. at an approximate ratio of 60.9%, 16.6%, and 20.4%, respectively). One unit of DPP-4 is the amount of enzyme that will hydrolyze the DPP-4 substrate to yield 1.0 µmole of AMC per minute at 37° C. A DPP-4 unit is equivalent to pmole/min/mL. GLP-1 blood levels are measured at different timepoints after ingestion and an optimal DPP-4 dosage is determined.

### Example 2: Dosage Forms

The following compositions according to this disclosure are prepared per the following table, in a manner known per se, e.g. by mixing the ingredients. The exemplary compositions are shelf stable. The materials are combined into a No. 0 or No. 00 immediate release capsule (e.g., Vcaps® Plus Capsugel®).

In Table 1 below, the alpha-glucosidase inhibiting component is an aqueous extract of Morus alba leaf and comprises 5 mass% of the iminosugar 1-deoxynojirimycin (DNJ). This component is commercially available as REDUCOSE®. The GLP-1 inhibiting component is DPP-4. The viscosity enhancing component is a soluble dietary fiber, e.g. guar gum. As these are merely example formulations of the inventive composition, no additional components are added.

TABLE 1

Exemplary Embodiments of the Oral Composition for the Treatment or Prevention of Dumping Syndrome

| | | | | | |
|---|---|---|---|---|---|
| Formulation: | A | B | C | D | E |
| Alpha-glucosidase inhibiting component | 250 mg | 125 mg | 500 mg | 225 mg | 125-500 mg |
| GLP-1 inhibiting component | 5,000 DPP-4 units | 500 DPP-4 units | 50,000 DPP-4 units | 5,000 DPP-4 units | 500-50,000 DPP-4 units |
| Viscosity enhancing component | 100 mg | 50 mg | 20 mg | 0 g | 0-100 mg |

### Example 3: Method of Treatment

The inventive composition is used on a patient who has undergone bariatric surgery, and who has dumping syndrome, to observe its effect. Patient comfort and glycemic/insulinemic response are determined without administering the composition at mealtime, and then with administration of the composition at mealtime. Patients receiving the composition report an improvement in general wellbeing, and/or experience fewer symptoms of dumping syndrome. Additionally, the composition reduces peak blood glucose levels (e.g. by 30%) and decrease the exaggerated postprandial GLP-1 and insulin increase.

### Example 4: Testing of Method of Use

A medical practitioner observes the inventive composition’s effect on a number of patients. Dumping symptoms improve after regular usage of the composition at each meal relative to not using the composition at each meal.

### Example 5: Study

A double-blind cross-over study to compare the inventive composition with standard treatment is summarized as follows. A double-blind, placebo controlled, cross-over study comparing the effects of the composition on dumping syndrome in bariatric patients suffering from dumping syndrome is conducted. Patients take the inventive oral composition or a control/placebo at the beginning of the meal (e.g. using a standardized meal).

At certain time points after food ingestion (e.g., 15 min, 30 min, 45 min, 1 h, 2 h, 4 h, ...) blood is drawn from patients for the determination of glucose, insulin and GLP-1 levels. Additionally, patients are asked to fill in a validated questionnaire regarding the presence of dumping symptoms.

The skilled artisan, using the directions herein and the known art is taught how to provide variations of this invention. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The following additional embodiments are provided, the numbering of which is not to be construed as designating levels of importance.

### ADDITIONAL EMBODIMENTS

Embodiment 1 relates to a dosage form (or composition) for the treatment of dumping syndrome, the composition comprising: a) alpha-glucosidase inhibiting portion, preferably an extract of Mulberry (Morus alba) leaf, more preferably an aqueous extract of Mulberry (Morus alba) leaf, most preferably an extract standardised to contain iminosugars of which about 5% m/m 1-deoxynojirimycin (DNJ); and b) a DPP-4 enzyme or fragment thereof, capable of effective degradation and inactivation of GLP-1.

Embodiment 2 relates Embodiment 1, wherein the glucosidase inhibiting portion is CAS 94167-05-02.

Embodiment 3 relates Embodiment 1 or Embodiment 2, wherein the composition additionally comprises a viscosity enhancing component.

Embodiment 4 relates to any one of the previous Embodiments, wherein dumping syndrome is addressed, and wherein the composition comprises additional vitamins and minerals that address post-operative nutritional deficiencies in bariatric surgery patients.

Embodiment 5 relates to any one of the previous Embodiments, wherein for bariatric surgery patients GI function or bowel health in bariatric surgery patients is improved.

Embodiment 6 relates to any one of the previous Embodiments, wherein the composition additionally comprises a viscosity enhancing component.

Embodiment 7 relates to any one of the previous Embodiments, wherein the composition additionally comprises a viscosity enhancing component and wherein the viscosity enhancing component comprises a soluble dietary fiber.

Embodiment 8 relates to any one of the previous Embodiments, wherein the composition additionally comprises vitamins and minerals.

Embodiment 9 relates to any one of the previous Embodiments, wherein the composition additionally comprises vitamins and minerals, selected from calcium, phosphorus, iron, chrome, copper, iodine, manganese, magnesium, molybdenum, selenium, sinc, vitamin B12, vitamin C, vitamins B1, B2, B3, B5, B6, B9, and fat soluble vitamins, preferably vitamins A, D, E, and K.

Embodiment 10 relates to any one of the previous Embodiments, wherein the composition additionally comprises vitamins and minerals, selected from calcium, phosphorus, iron, vitamin B12, vitamin C and fat soluble vitamins, selected from vitamin A, vitamin D, vitamin E, and vitamin K.

Embodiment 11 relates to any one of the previous Embodiments, wherein for the composition, a per meal dosage of the mulberry leaf extract as specified before is from about 125 mg to 500 mg per dose, more preferably 200 to 300 mg per dose, most preferably 225 to 250 mg per dose.

Embodiment 12 relates to any one of the previous Embodiments, wherein the viscosity enhancing additive in the per meal dosage of the composition is from about 0.05 g to 20 g per dose, more preferably 50 mg to 15 g per dose, most preferably 100 mg to 5 g per dose.

Embodiment 13 relates to any one of the previous Embodiments, wherein the composition is useful in treating dumping syndrome.

Embodiment 14 relates to any one of the previous Embodiments, wherein the composition is used in treating dumping syndrome related to bariatric surgery.

Embodiment 15 relates to any one of the previous Embodiments, directed toward use of the composition for the manufacture of a medicament for treating or preventing dumping syndrome.

Embodiment 16 relates to any one of the previous Embodiments, directed toward a method of treating or preventing dumping syndrome using the composition.

Embodiment 17 relates to any one of the previous Embodiments, directed toward a method of treating or preventing dumping syndrome using the composition wherein the composition or dose is administered immediately before or with a meal.

Embodiment 18 relates to any one of the previous Embodiments, directed toward a method of treating or preventing dumping syndrome associated with Roux-en-Y gastric bypass and vertical sleeve gastrectomy, using the composition wherein the composition or dose is administered immediately before or with a meal.

Embodiment 19 relates to any one of the previous Embodiments, wherein the dosage form of the composition comprises 225 mg of a glucosidase inhibiting portion, wherein the glucosidase inhibiting portion is a Mulberry leaf extract known as CAS 94167-05-02 and 5,000 units of DPP-4.

The terms “comprising” or “comprise” are used herein in their broadest sense to mean and encompass the notions of “including,” “include,” “consist(ing) essentially of,” and “consist(ing) of.” The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-25, ±0-10, ±0-5, or ±0-2.5, % of the numerical values. Further, The term “about” applies to both numerical values when associated with a range of values. Moreover, the term “about” may apply to numerical values even when not explicitly stated.

Generally, as used herein a hyphen “-” or dash “-” in a range of values is “to” or “through”; a “>” is “above” or “greater-than”; a “≥” is “at least” or “greater-than or equal to”; a “<” is “below” or “less-than”; and a “≤” is “at most” or “less-than or equal to.” On an individual basis, each of the aforementioned applications for patent, patents, and/or patent application publications, is expressly incorporated herein by reference in its entirety in one or more nonlimiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

### CITATIONS LIST

1. Papamargaritis D, et al. (2012) Dumping symptoms and incidence of hypoglycaemia after provocation test at 6 and 12 months after laparoscopic sleeve gastrectomy. Obes Surg 22(10):1600-1606.

2. Tack J, Arts J, Caenepeel P, De Wulf D, & Bisschops R (2009) Pathophysiology, diagnosis and management of postoperative dumping syndrome. Nat Rev Gastroenterol Hepatol 6(10):583-590.

3. Tzovaras G, et al. (2012) Symptoms suggestive of dumping syndrome after provocation in patients after laparoscopic sleeve gastrectomy. Obes Surg 22(1 ):23-28.

4. Emous M, Wolffenbuttel BHR, Totte E, & van Beek AP (2017) The short- to mid-term symptom prevalence of dumping syndrome after primary gastric-bypass surgery and its impact on health-related quality of life. Surg Obes Relat Dis 13(9):1489-1500.

5. Cadegiani FA & Silva OS (2016) Acarbose promotes remission of both early and late dumping syndromes in post-bariatric patients. Diabetes Metab Syndr Obes 9:443-446.

6. Scarpellini E, et al. (2020) International consensus on the diagnosis and management of dumping syndrome. Nat Rev Endocrinol.

7. Holst JJ (2007) The physiology of glucagon-like peptide 1. Physiol Rev 87(4):1409-1439.

8. Peterli R, et al. (2012) Metabolic and hormonal changes after laparoscopic Roux-en-Y gastric bypass and sleeve gastrectomy: a randomized, prospective trial. Obes Surg 22(5):740-748.

9. Nosso G, et al. (2016) Comparative Effects of Roux-en-Y Gastric Bypass and Sleeve Gastrectomy on Glucose Homeostasis and Incretin Hormones in Obese Type 2 Diabetic Patients: A One-Year Prospective Study. Horm Metab Res 48(5):312-317.

10. Salehi M, Gastaldelli A, & D′Alessio DA (2014) Blockade of glucagon-like peptide 1 receptor corrects postprandial hypoglycemia after gastric bypass. Gastroenterology 146(3):669-680 e662.

11. Salehi M, Prigeon RL, & D′Alessio DA (2011) Gastric bypass surgery enhances glucagon-like peptide 1-stimulated postprandial insulin secretion in humans. Diabetes 60(9):2308-2314.

12. Craig CM, Liu LF, Deacon CF, Holst JJ, & McLaughlin TL (2017) Critical role for GLP-1 in symptomatic post-bariatric hypoglycaemia. Diabetologia 60(3):531-540.

13. Alam ML, et al. (2011) Gastric bypass surgery, but not caloric restriction, decreases dipeptidyl peptidase-4 activity in obese patients with type 2 diabetes. Diabetes Obes Metab 13(4):378-381.

14. Lown M, et al. (2017) Mulberry-extract improves glucose tolerance and decreases insulin concentrations in normoglycaemic adults: Results of a randomised double-blind placebo-controlled study. PLoS One 12(2):e0172239.

15. Valderas JP, et al. (2012) Acarbose improves hypoglycaemia following gastric bypass surgery without increasing glucagon-like peptide 1 levels. Obes Surg 22(4):582-586.

16. Andersen JR, Holtug K, & Uhrenholt A (1989) Trial of pectin-enriched muffins in patients with severe dumping syndrome after gastric resection. Observations on symptoms and gastric emptying pattern. Acta Chir Scand 155(1):39-41.

17. Harju E, Heikkila J, & Larmi TK (1984) Effect of guar gum on gastric emptying after gastric resection. JPEN J Parenter Enteral Nutr 8(1 ):18-20.

18. Harju E & Larmi TK (1983) Efficacy of guar gum in preventing the dumping syndrome. JPEN J Parenter Enteral Nutr 7(5):470-472.

19. Jenkins DJ, et al. (1980) Pectin and complications after gastric surgery: normalisation of postprandial glucose and endocrine responses. Gut 21 (7):574-579.

20. Jenkins DJ, et al. (1977) Effect of dietary fiber on complications of gastric surgery: prevention of postprandial hypoglycemia by pectin. Gastroenterology 73(2):215-217.

21. Kneepkens CM, Fernandes J, & Vonk RJ (1988) Dumping syndrome in children. Diagnosis and effect of glucomannan on glucose tolerance and absorption. Acta Paediatr Scand 77(2):279-286.

22. Leeds AR, Ralphs DN, Ebied F, Metz G, & Dilawari JB (1981) Pectin in the dumping syndrome: reduction of symptoms and plasma volume changes. Lancet 1(8229):1075-1078.

What is claimed is:

1. An oral composition for the treatment of dumping syndrome, the oral composition comprising:
a) an alpha-glucosidase inhibiting component comprising an extract of Morus alba; and
b) a glucagon-like peptide (GLP-1) inhibiting component comprising a dipeptidyl peptidase-4 (DPP-4) enzyme or fragment thereof.

2. The oral composition according to claim 1, wherein the extract of Morus alba is an aqueous extract of Morus alba leaf.

3. The oral composition according to claim 2, wherein the aqueous extract of Morus alba leaf is CAS 94167-05-2.

4. The oral composition according to claim 2, wherein the aqueous extract of Morus alba leaf comprises an iminosugar in an amount of from 2.5 to 15 mass%.

5. The oral composition according to claim 1, wherein the dipeptidyl peptidase-4 (DPP-4) enzyme or fragment thereof is present in an amount of from 300 to 60,000 DPP-4 units.

6. The oral composition according to claim 1, additionally comprising a viscosity enhancing component.

7. The oral composition according to claim 6, wherein the viscosity enhancing component comprises a soluble dietary fiber.

8. The oral composition according to claim 6, wherein the viscosity enhancing component comprises at least one of gum, fiber, pectin, polysaccharide, starch, or combinations thereof.

9. The oral composition according to claim 1, additionally comprising a supplement component.

10. The oral composition according to claim 9, wherein the supplement component comprises a least one of calcium, phosphorus, iron, chrome, copper, iodine, manganese, magnesium, molybdenum, selenium, zinc, vitamin B12, vitamin C, vitamins B1, B2, B3, B5, B6, B9, A, D, E, or K, or combinations thereof.

11. The oral composition according to claim 1, wherein the extract of Morus alba is present in an amount of from 125 mg to 500 mg.

12. The oral composition according to claim 11, wherein the extract of Morus alba is present in an amount of from 200 mg to 300 mg.

13. The oral composition according to claim 12, wherein the extract of Morus alba is present in an amount of from 225 mg to 250 mg.

14. The oral composition according to claim 6, wherein the viscosity enhancing component is present in an amount of from 0.01 g to 20 g.

15. The oral composition according to claim 14, wherein the viscosity enhancing component is present in an amount of from 0.05 g to 15 g.

16. The oral composition according to claim 15, wherein the viscosity enhancing component is present in an amount of from 0.05 g to 5 g.

17. A medicament for treating or preventing dumping syndrome, wherein the medicament comprises the oral composition according to claim 1.

18. A method of treating or preventing dumping syndrome, the method comprising:
providing the oral composition according to claim 1; and
orally administering the oral composition to a patient.

19. The method according to claim 18, wherein the oral composition is administered immediately before or with a meal.

20. The method according to claim 19, wherein orally administering the oral composition further comprises mixing the oral composition with a component of the meal.
